(19) United States
(12) Patent Application Publication
(10) Pub. No.: US 2008/0260229 A1
(43) Pub. Date: Oct. 23, 2008

(54) System and method for segmenting structures in a series of images using non-iodine based contrast material

(76) Inventor: Adi Mashiach, Tel Aviv (IL)

Correspondence Address: EMPK & Shiloh, LLP;c/o Landon IP, Inc., 1700 Diagonal Road, Suite 450, Alexandria, VA, 22314, US

(21) Appl. No.: 11/802,928

(22) Filed: May 25, 2007

Related U.S. Application Data
(60) Provisional application No. 60/808,129, filed on May 25, 2006.

(51) Int. Cl. A61B 5/00

(52) U.S. Cl. 382/131

(57) ABSTRACT

A method and system of defining a boundary of a part of a blood vessel in an image captured by an ex vivo imager, where such part of the blood vessel is free from iodine-based contrast material.

### REFERENCE TO RELATED APPLICATIONS

This application claims the benefit of U.S. Provisional Application No. 60/808,129 filed on May 25, 2006 and entitled ‘System and Method for Segmenting Structures in a Series of Images Using Non-Iodine Based Contrast Material’, which is incorporated herein by reference in its entirety.

### BACKGROUND OF THE INVENTION

Capturing images of internal areas, structures or organs of a body may include administering contrast material to for example highlight the areas or organs being imaged. When imaging for example blood vessels, a contrast material may be injected into the circulatory system so that the shape, path or outline of a vessel being imaged is highlighted in an image. Contrast material may also be administered when imaging for example an alimentary canal, excretory organs or other tubular organs. Some patients may be allergic to iodine based contrast materials. Some iodine based contrast materials may have adverse effects on patients such as for example patients with renal complications.

### BRIEF DESCRIPTION OF THE FIGURES

The subject matter regarded as the invention is particularly pointed out and distinctly claimed in the concluding portion of the specification. The invention, however, both as to organization and method of operation, together with features and advantages thereof, may best be understood by reference to the following detailed description when read with the accompanied drawings in which:

FIG. 1 is a schematic diagram of an image processing device and system, in accordance with an embodiment of the invention;

FIG. 2 is a depiction of a series of images of a body part captured by an ex vivo imager, in accordance with an embodiment of the invention;

FIG. 3 is a schematic depiction of a segmented vessel, in accordance with an embodiment of the invention;

FIG. 4 is a flow diagram of a method, in accordance with an embodiment of the invention;

FIGS. 5A and 5B are depictions of isolable contour level regions in an embodiment of the invention;

FIG. 5C is a depiction of neighborhoods of pixels in areas between edges of isolable contour regions in an embodiment of the invention; and

FIG. 5D is a flow diagram of a method of clustering pixels into ranges of intensity levels and mapping contour levels of the clustered pixels to segment a body part in an image, in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various embodiments of the invention will be described. For purposes of explanation, specific examples are set forth in order to provide a thorough understanding of at least one embodiment of the invention. However, it will also be apparent to one skilled in the art that other embodiments of the invention are not limited to the examples described herein. Furthermore, well-known features may be omitted or simplified in order not to obscure embodiments of the invention described herein.

Unless specifically stated otherwise, as apparent from the following discussions, it is appreciated that throughout the specification, discussions utilizing terms such as “selecting,” “processing,” “computing,” “calculating,” “determining,” or the like, may refer to the actions and/or processes of a computer, computer processor or computing system, or similar electronic computing device, that may manipulate and/or transform data represented as physical, such as electronic, quantities within the computing system's registers and/or memories into other data similarly represented as physical quantities within the computing system's memories, registers or other such information storage, transmission or display devices. In some embodiments processing, computing, calculating, determining, and other data manipulations may be performed by one or more processors that may in some embodiments be linked.

In some embodiments, the term ‘free of contrast material’ or ‘not highlighted by contrast material’ may, in addition to the regular understanding of such term, mean having a non-iodine based contrast material. For purposes of this paper, a vessel into which has been administered Gadolinium chelates or other non-iodine based contrast material is considered to be a vessel that is free of iodine contrast material or that does not have an iodine based contrast material present.

The processes and functions presented herein are not inherently related to any particular computer, imager, network or other apparatus. Embodiments of the invention described herein are not described with reference to any particular programming language, machine code, etc. It will be appreciated that a variety of programming languages, network systems, protocols or hardware configurations may be used to implement the teachings of the embodiments of the invention as described herein.

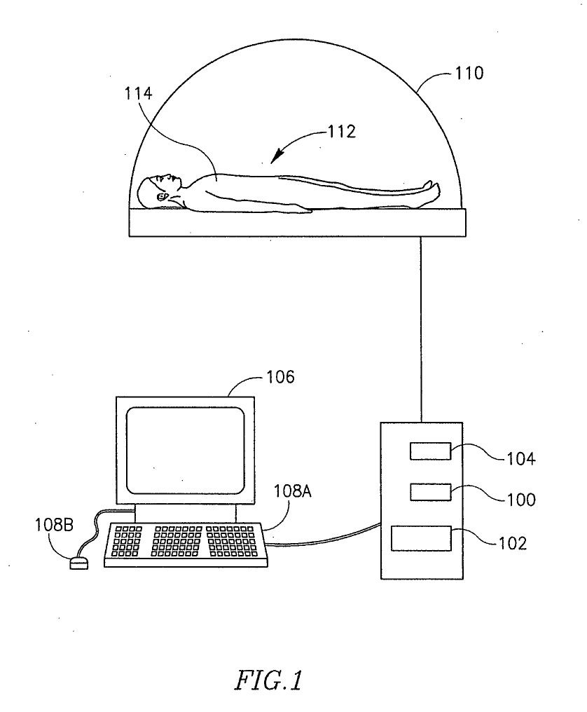

Reference is made to FIG. 1, a schematic diagram of an image processing device and system, in accordance with an embodiment of the invention. An image processing device in accordance with an embodiment of the invention may be or include a processor 100 such as for example a central processing unit. The image processing device may include or be connected to a memory unit 102 such as a hard drive, random access memory, read only memory or other mass data storage unit. In some embodiments, processor 100 may include or be connected to a magnetic disk drive 104 such as may be used with a floppy disc, disc on key or other storage device. The image processor may include or be connected to one or more displays 106 and to an input device 108 such as for example a key board 108A, a mouse, or other pointing device 108B or input device by which for example, a user may indicate to a processor 100 a selection or area that may be shown on a display. In some embodiments, processor 100 may be adapted to execute a computer program or other instructions so as to perform a method in accordance with embodiments of the invention.

The processor 100 may be connected to an external or ex vivo diagnostic imager 110, such as for example a computerized tomography (CT) device, ultrasound scanner, CT Angiography, magnetic resonance angiograph, positron emission tomography or other imagers 110. In some embodiments, imager 110 may capture one or more images of a body 112 or body part such as for example a blood vessel 114, a tree of blood vessels, alimentary canal, urinary tract, reproductive tract, or other tubular vessels or receptacles. In some embodiments imager 110 or processor 100 may combine one or more images or series of images to create a 3D image or volumetric data set of an area of interest of a body or body part such as for example a blood vessel 114. In some embodiments, a body part may include a urinary tract, a reproductive tract, a bile duct, nerve or other tubular part or organ that may for example normally be filled or contain a body fluid. In some embodiments, imager 110 and/or processor 100 may be connected to a display 106 such as a monitor, screen, or projector upon which one or more images may be displayed or viewed by a user.

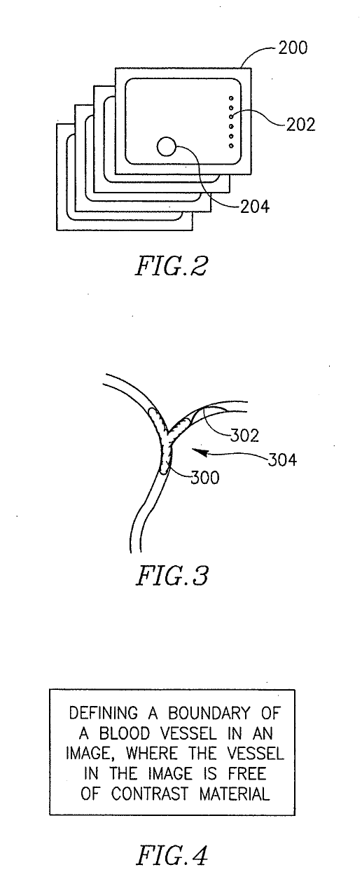

Reference is made to FIG. 2, a depiction of a series of images in accordance with an embodiment of the invention. In some embodiments, a series of images 200 may be arranged for example in an order that may, when such images 200 are stacked, joined or fused by for example a processor, create a three dimensional view of a body part such as a blood vessel 114, or provide volumetric data on a body part or structure. In some embodiments images 200 in a series of images may be numbered sequentially or otherwise ordered in a defined sequence. In some embodiments, images 200 may include an arrangement, matrix or collection of pixels 202, voxels or other atomistic units that may, when combined create an image. In some embodiments, pixels 202 may exhibit, characterize, display or manifest an image intensity of the body part appearing in the area of the image 200 corresponding to the pixel 202. In some embodiments, an image intensity of a pixel 202 may be measured in Hounsfield units (HU) or in other units.

In some embodiments, a location of a pixel 202 in an image 200 may be expressed as a function of coordinates of the position of the pixel on a horizontal (x) and/or vertical (y) axis. Other expressions of location, intensity and characteristics may be used.

In some embodiments, a user of an image processing device or system may view an image 200 on for example display 106, and may point to or otherwise designate an area of the image 200 as for example a seed area 204. In some embodiments, a seed area 204 may be or include a location within an image 200 of a body part such as for example a vessel 114 or other structure or organ in a body. In some embodiments, a seed area 204 may include one or more pixels and a description of, or data about, a body part or organ that may appear in an image or series of images, such as the image intensity of pixels 202 in such seed area 204.

Reference is made to FIG. 3, a schematic depiction of a vessel 304 segmented from surrounding structures, in accordance with an embodiment of the invention. In some embodiments, a contrast material 300 such as Gadolinium Chelates or other non-iodine based contrast material, may be administered by way of for example ingestion, injection or otherwise into a body part such as vessel 304. In some embodiments, a calcified substance on an area of a vessel or vessel wall may be highlighted in an image.

Reference is made to FIG. 4, a flow diagram of a method in accordance with an embodiment of the invention. In block 400, an image processor may define a boundary of a vessel or part of a vessel in an image or series of images, where the vessel in the image is not filled with contrast material, or is filled with Gadolinium Chelates or other non-iodine based contrast material. In some embodiments of the invention, an image processor may segment, trace, define, display, differentiate, identify, measure, characterize, make visible or otherwise define a vessel or part of a vessel that contains Gadolinium Chelates or other non-iodine based contrast material. In some embodiments, an image processor may display or define one or more boundaries, edges, walls or characteristics such as diameter, thickness of a wall, position, slope, angle, or other data of or about an organ or vessel when such vessel is highlighted by Gadolinium Chelates or other non-iodine based contrast material. Other boundaries or characteristics of a vessel may be displayed or defined in for example an image or in other collections of data about the vessel.

In operation, an image processor in an embodiment of the invention may map, depict or segment an organ or vessel in an image by clustering pixels in an area of interest of an image into a number of clusters of image intensity levels. The range of image intensity levels of pixels that may be included in a cluster may include variably or unevenly sized ranges of image intensities, such that the ranges of intensity levels in a cluster are non-uniform. In some embodiments, numerous clusters of pixels in a range of image intensity levels may be created in the levels that generally appear in images of soft tissue, while other kinds of tissue may be represented by fewer clusters. In some embodiments, certain of the clusters may be disregarded in an image as not being part of or related to the target organ or vessel. Prior to removing or disregarding clusters, some pixels that were not mapped to these clusters may in some embodiments be added, in for example a bottom hat operation. In some embodiments, adding such pixels may be accomplished by transforming the pixels in the cluster that would otherwise have been disregarded into an image, and applying a closing operation or another morphological operation to such image. The added pixels may then be disregarded along with the pixels in a cluster that is disregarded. Other processes may be used.

In some embodiments, certain clusters of pixels may be mapped into regions of isolable contour levels, where a mapped region shows an area of a cluster of pixels having a given range of image intensities. In some embodiments, the isolable contour region that has a range of image intensity levels which is the same as or similar to the range of image intensity levels of a seed area, and /or whose area overlaps or may be in contact with a seed area of for example a prior image, may be identified as including the target vessel. In some embodiments, an area selected as including the target vessel in an image may be designated as a seed area in a succeeding image.

In some embodiments, a selection of one among a plurality of possible isolable contours regions that may define or enhance the accuracy of one or more boundaries of a target vessel, may be made by for example comparing geometric characteristics of a view of for example a target vessel or other area as it is presented in for example two or more isolable contour regions. In some embodiments, the accuracy of the selection of an isolable contour region that defines a boundary of a target vessel may also be checked through texture analysis of a target vessel as the vessel is presented in various images having areas of interests of different sizes. In some embodiments, the sharpness or definition or accuracy of definition of a target vessel or organ identified in a segmentation may be checked, optimized or improved by first standardizing the size or number of pixels in a particular area of interest of an image, such as for example a seed area by for example standardizing the entropy figure by diving the entropy figure by a log of the number of pixels in the area whose entropy is measured, and comparing the standardized entropy of an area of interest in a first image to a standardized entropy in an area of interest of another image.

In some embodiments, more than one target vessel or organ may be serially or concurrently segmented in an image or series of images, and a processor may recursively segment the selected vessels beginning at the various seed points, as such points may have been indicated by for example a user in a first or successive image. In some embodiments, a memory may record the seed points and the vessels and branches that may extend from such points.

In some embodiments, data and coordinates of a location, plane, orientation and dimensions of a target vessel may be stored as x, y, z binary 3D volume data, where a 3D matrix indicates pixels belonging to the segmented vessel. The result matrix may be further processed, for instance by morphological operations such as the deletion of elements below a certain predefined number of pixels or the filling of holes in a segmented slice by applying a flood-fill operation on a set of background pixels unreachable from the edges of the vessel slice.

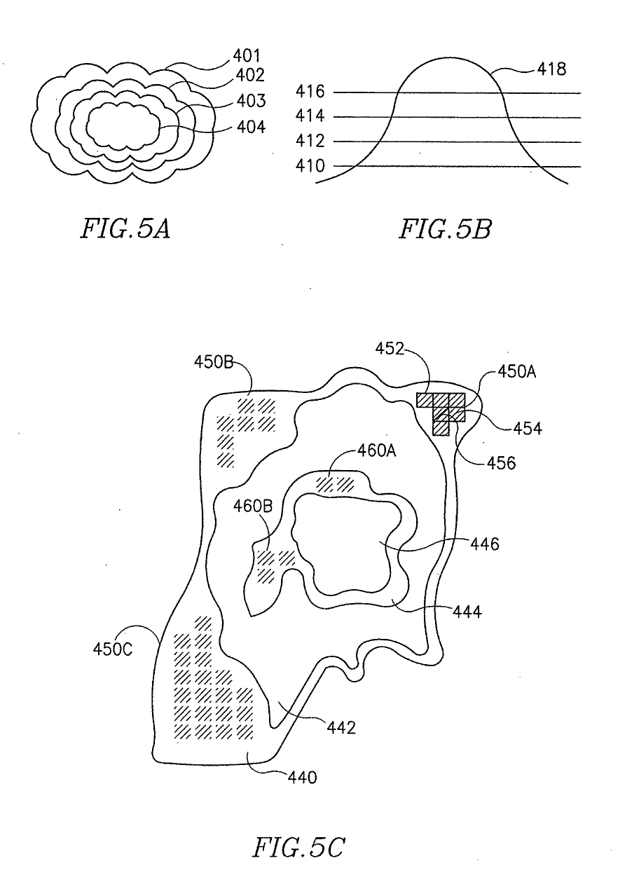

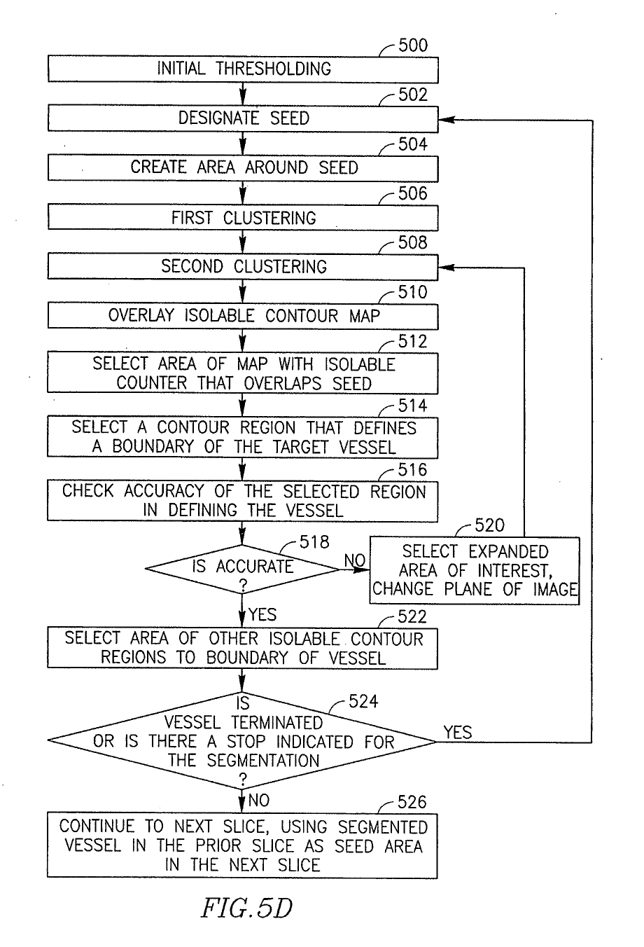

Reference is made to FIG. 5A, a schematic depiction of isolable contour regions defining areas of image intensities of pixels in accordance with an embodiment of the invention. In some embodiments, an isolable region 401 with a lowest level may define an area of pixels having an intensity level of at least for example −1000 Hu. A another isolable contour region 402 may define an area that includes pixels having intensities of at least −100 Hu, another intensity level may define an area with pixels having intensity levels of at least 0 Hu, another isolable region may define an area having pixels with for example 60 Hu, another isolable region 403 may define an area having pixels with for example 300 Hu, and another isolable region 404 may define an area having pixels with for example 800 Hu. Not all isolable regions may be shown in FIG. 5A. In some embodiments, a boundary of a target vessel may be defined by the limits of one or more isolable contour regions. In some embodiments, a boundary of a target vessel may include data on a circumference, area and position of the target vessel.

Reference is made to FIG. 5D, a flow diagram of a method of clustering pixels into ranges of intensity levels and mapping contour levels of the clustered pixels to segment a body part in an image in accordance with an embodiment of the invention. In block 500, and in some embodiments, an initial threshholding of an image or area of an image may highlight possible areas of interest or desired characteristics in possible areas of interest. For example, an initial threshholding may remove pixels or areas of pixels having image intensities of less than a defined Hu level. Such a defined level may approximate an image intensity level of pixels that correspond to for example areas of water or air or other items in an image that are not of interest to a particular segmentation exercise.

In some embodiments, the image used for designating an area of interest may be a first image in a series of images. In some embodiments a starting point in a segmentation in accordance with an embodiment of the invention may begin at a last or intermediate slice in a series of images and may proceed to a first, previous or later slice. In some embodiments, segmentation may proceed in both directions out from a particular starting slice. In some embodiments, a segmentation in an embodiment of the invention may move in a coronal, sagital or other plane of a body, organ, vessel or other structure. Other slices or images in a series of images or orders of segmentation of such images may be used. A direction, order, vector or plane of images may be altered in one or more processes of segmenting a vessel.

In some embodiments, an image intensity threshold for an initial threshholding may be designated by a user in for example an iterative process where a user may highlight a possible area of interest, and reject pixels below a threshold intensity level that approximates the intensity level of pixels of the target vessel. In some embodiments, an image intensity level for an initial threshholding may be designated by for example a processor, based on for example data about the organ or target vessel to be segmented. For example, a user may identify a vessel or structure by for example, name, region, thickness or other characteristics. A processor may reference a data base that may include for example historic samples of for example grayscale scorings of an identified vessel, shapes of an identified vessel or average image intensities of the identified vessels that are being targeted for segmentation. The processor may threshold intensities that are out of a range of such sampled or average intensity levels or may otherwise locate the target vessel in for example a first image

A result of the initial threshholding may be a highlighting or designation of an area of interest that may include the target vessel or organ. Other methods may be used to select an area of interest in an image. In some embodiments an area of interest may be smaller than or may include fewer pixels than the entire area of the image. In some embodiments an entire area of an image may be designated as an area of interest.

In block 502 a seed point or seed area may be selected or designated in the area of interest of an image. In some embodiments a seed area may be co-extensive with an area of interest. In some embodiments, a user may select a seed point or seed area by way of pointing to or otherwise indicating the selected seed point or area with a pointing device that may for example be connected to a display. In some embodiments, a seed point may be selected automatically by for example a processor based on the input by for example a user of data on a target vessel to be segmented. Such data may be or include for example a name of a vessel or organ, a shape of the target vessel or organ, an expected image intensity of the target vessel or organ or other data. In some embodiments, a processor may reference a data base of, for example, sample image intensity data or shapes of a particular vessel or organ, and compare such historic data with the displayed image to locate and select the named vessel or organ. Other methods of selecting or designating a seed point target vessel in an image are possible.

A seed point may be or include one or more pixels within the seed area. For example, a processor may select a seed point as a mass center of a region, or as a pixel with an image intensity value having a mean, median or average of the image intensities of pixels present in the specified area. In some embodiments, a user may select a seed point or area. Other methods of selecting a seed point or area are possible.

In some embodiments, coordinates of the seed point may be recorded. Such coordinates may include for example horizontal (x) and vertical (y) coordinates in the image of one or more pixels in the seed point, a slice or image number of the image in the series of images (z) and an image intensity (v) or average, median mean or other intensity characteristic of one or more pixels in or around the seed point.

In some embodiments, coordinates of for example a seed point may include data regarding a plane upon which sits the image wherein the seed was identified Other coordinates or characteristics of a seed point or seed area may be recorded or stored.

In some embodiments one or more seed points may be selected within an image, and a processor may serially, recursively or consecutively segment one or more target vessels in such image. Other methods or orders of segmenting multiple seeds or branch list stacks are possible.

In block 504, and in some embodiments, a seed area may be selected or designated around for example a seed point. In some embodiments, dimensions of an area of interest may be selected to create for example a bounding box, a convex hole or other shapes around a seed point or group of pixels surrounding a seed point. Other shapes may be used to surround or designate a seed area or area of interest. In some embodiments, for example a radii, diagonal or other measure of a bounding box, convex hole, circle or other shape around a seed area or seed point may be multiplied, divided or otherwise changed by for example a factor of two, three or some other factor to approximate the likely areas wherein the target vessel may be found in the image or in a subsequent image in the series of images. Other factors or processes for creating an area of interest may be used such as for example log or others.

Refining or adjusting the size, shape or location of an area of interest may improve the likelihood that the area of interest includes the likely dimensions of the target vessel without encompassing unnecessary additional areas. An area of interest that is too large may include too may gray-scale levels which may reduce the effectiveness of clustering. An area of interest that is too small may not include the boundaries of a target vessel being segmented.

In block 506, and in some embodiments, a first clustering of pixels in the area of interest may be performed. In some embodiments a first clustering may designate for example four or several image intensity levels (N1) as for example cluster center means, and may create several clusters of pixels around such centers. In some embodiments, the clusters corresponding to a lowest image intensity level that may correspond to imaged items that are not of interest, such as air and water, may be excluded from further clustering.

In block 508 and in some embodiments, a second clustering may be performed on the pixels that are in the intensity levels that were not excluded in the first clustering. In such clustering, a larger or much larger number of image intensity levels (N2) may be selected as cluster centers, such as 6, 10 or even more. In some embodiments, the number of clusters that may be selected may be a function of the processing power and time that may be available to complete a clustering process. Other functions for determining a number of clusters are possible.

A second clustering may separate pixels into a large number of clusters based on relatively small differences in the image intensity of such pixels. For example, a user may instruct a processor to create 15 clusters that may include varying sized ranges of intensity levels. In some embodiments, a processor may automatically select one or more clusters center means, and the range of one or more of clusters to be created around such centers, based on for example an intensity of a seed point or an average intensity of a seed area. For example if an intensity level of a seed point is high, a set of cluster center means may be selected in a relatively high range on a pixel intensity scale. If an intensity level of a seed point or seed area is relatively low, a different set of possible cluster center means in for example a lower area or range of the intensity scale may be chosen. Other criteria may be used to select a number of clusters and a set of cluster center means. In some embodiments, pixels may be clustered by characteristics other than their image intensity levels, or by a combination of image intensity levels and other characteristics.

In some embodiments, the range of intensity units in a cluster may be variable or non-uniform, such that the intensity levels may be un-evenly spaced along the range of possible intensity units that may be relevant to an area of interest.

In some embodiments, a user or a processor may select the cluster center means of one or more clusters. Selection of a cluster center mean with for example an image intensity that is present in for example a target vessel may facilitate differentiating a target vessel from surrounding structures. In some embodiments, several possible groups or sets of cluster centers may be defined by for example a processor, one for example for normal contrast intensities, and a second for low contrast intensities. A set of possible cluster center means may be assembled by a user or selected from a pre-defined list. In some embodiments, one or more cluster center means may be selected based on a range of image intensities in a seed area, such that the cluster center means is similar to the range of image intensity levels in the seed area.

In some embodiments, an increase in the number of ranges of intensity levels that may be selected and in the number of clusters that are created, may increase the differentiation that is possible of pixels that have relatively similar image intensities. For example, increasing the number of clusters and, for example, setting one or more cluster center means to the image intensity level of a vessel wall, and another cluster center to the image intensity level of for example a blockage, plaque or other material that may adhere to or extend from a vessel wall, may highlight differences between an inner wall of a vessel and a blockage near such wall. In some embodiments, the number of intensity units that are included in a range of intensity levels used for clustering may be variable or different than the number of intensity units included in another level, such that the intensity levels may be un-evenly spaced along the range of intensity units in the area of interest.

In some embodiments, clustering may include a fuzzy c-means clustering process. In some embodiments clustering may include a k-means clustering. Other methods of clustering are possible.

In some embodiments, where for example, two or more cluster areas of similar image intensities appear in an image, a cluster area may be selected as the probable target vessel based on for example a distance of the cluster area from the seed area in for example a prior image. For example, where in an image there appear two or more cluster areas having a same or similar cluster center means, the area that is closest to a seed area of a prior image may be selected as the most likely target vessel. Other processes for selecting a probable cluster as representing a target vessel are possible such as multiplying, for example and several methods of calculating a distance transform may be applied, for example a Euclidean distance transform.

In block 510, an isolable contour map may be overlaid on the image so that the contours correspond to the location of the various clusters of the pixels in the area of interest on the image. Reference is made to FIG. 5A, which depicts a conceptual representation of isolable contours overlaid over a group of pixels. In some embodiments a contour level may surround pixels in an area, where the encompassed pixels have image intensities of at least a certain level (Hu1). Another contour level may encompass pixels in for example a smaller area within the prior level, where such pixels have image intensities of at least a certain level (Hu2), where Hu2>Hu1. A next contour level may sit within the prior contour level, and may encompass pixels in a still smaller area where the encompassed pixels have image intensities of at least a certain level (Hu3), (Hu3>Hu2>Hu1). A highest contour level may encompass pixels in an area where the encompassed pixels have image intensities of for example a highest level in the relevant area. The resulting contour map may in some embodiments not contain empty matrices or contour levels that display higher image intensities than those that are present in the relevant area of the image. FIG. 5B is a conceptual depiction of a side view of mapped isolable contour regions, where lines 410, 412, 414 and 416 represent for example end points of image intensity ranges that may be included in a cluster and curve 418 represents the encompassed area of the mapped isolable contour areas of a target vessel. Other designations or measures of intensity levels are possible.

In some embodiments a color or other marking may be assigned to a contour level and such color may appear on a display of an image in the area of the contour. In some embodiments, various colors or other display characteristics may be assigned to each contour that is displayed.

In block 512, there may selected an area of the overlaid map that has a contour level region of image intensities that for example matches an intensity level of a seed point or seed area and, that for example includes or overlaps at least one pixel from a seed point or seed area. In some embodiments, there may be excluded contour areas whose range of pixel intensities may match the image intensity level of the contour that includes the seed point, but that do not have contact with or overlap the seed area in an image, or in a prior image. Such exclusion of non-overlapping contour areas may exclude from the further segmentation process images of for example other vessels in the area of interest that may have the same or similar image intensity levels as the target vessel but that are not the target vessel. For example, a contour map of an area of interest of an image may include two contour areas with image intensities that match the 60-300 Hu level of the seed point in the image or in a prior image slice. In some embodiments, a processor or user may select for continued segmentation only the isolable contour level region with for example a matching intensity level and whose area overlaps or is otherwise in contact with the seed point of the image or of a prior image. This overlapping contour may likely include the target vessel in the image. In some embodiments, a selection of an overlapping contour may be achieved with an AND bitwise operator.

In block 514, and in some embodiments, a determination may be made of a contour level region that most closely defines a boundary of a target vessel. For example, and referring to FIG. 5A, the selection of contour 401 as presenting a view of a target vessel, may indicate a much wider vessel than a selection of contour level 404.

In some embodiments, an evaluation of the shape or other geometric properties of contour level regions may be used to determine a contour level that most closely defines a boundary of a target vessel. One such evaluation may include a comparison of shapes or other geometric properties of the areas of pixels encompassed by the various contour regions depicted on the overlaid map. Such a comparison may include calculating a minimum derivative of ΔAD, where ΔAD=ΔArea*ΔDistance, where ΔArea is the change in the total area between two isolable contour regions in a clustered area of an image, and ΔDistance is the distance along the x and y axis between a center of mass of such two isolable contour regions. In some embodiments, contour region i+1 may be selected, where i is the contour region in respect of which ΔAD crosses the x axis to denote a zero change in ΔAD between the relevant contour regions. For example, and returning to FIG. 5A, if in a comparison of contour region 401 and contour region 402, ΔAD is zero, contour region 402 may be selected as defining a boundary of a target vessel. If there is more than one phase of ΔAD crossing the x axis, the first point in the second phase may be selected. Other methods of selecting a contour region that defines a boundary of a target vessel may be used.

In some embodiments, a texture analysis or comparison of entropy dimensions of areas of pixels encompassed by the various contour regions may be used to select a contour region that defines a boundary of a target vessel and/or to evaluate the accuracy or suitability of a contour level region that was selected as defining a boundary of a target vessel. A texture analysis using entropy dimensions may assume that the appearance in an image of pixels that are not part of a target vessel will have a higher entropy dimension (De) than a pre-defined threshold, and that the appearance of too few pixels will have a lower De than such threshold. A contour region may be varied and an entropy dimension of the image regarded as a fractal may be evaluated for one or more of the isolable contour regions. For example, if the intensity level of the cluster of the isolable contour region was too low, then too many pixels may be included in the region defining the target vessel, and a next higher contour level region may better define the boundaries of the vessel. If the intensity level was too high, then too few pixels may be included in such region, and a lower contour level region may be more appropriate for defining the vessel.

In some embodiments the two or more contour regions or areas of interests whose De is to be compared may have different areas and different number of pixels. A standardizing function, such as for example (De value−De minimum)/(De maximum−De minimum) may standardize the De between the two regions so that the De values can be meaningfully compared. In some embodiments, a De of the compared regions may be standardized with the log of the number of pixels in each of the regions, as follows, Standardized De (SDe)=De/log(N), where N is the number of pixels in the part of the respective image whose De is being evaluated. In such case, SDe of a first image may be meaningfully compared to SDe of a second image or to a threshold level. In some embodiments a threshold range for SDe may be from 0.17 to 0.05, such that if a comparison of areas of interest or isolable regions yields an SDE within such range, the contour region or area of interest with the higher image intensity level may be selected as defining the target vessel or including the target vessel. In some embodiments, the region or area of interest selected may be the one with the lowest SDe value or the one with the closest value of SDe to a predefined value. Other threshold ranges may be used, and other methods of selecting an isolable contour region or area of interest may be used.

In some cases, an SDe of a contour region having even a lowest image intensity range of clustered pixels may be out of an acceptable SDe range. Such result may be caused by for example, the target vessel filling or taking up the entire area of interest that had been clustered, or by the disappearance of the target vessel from the particular image. To determine whether a target vessel takes up the entire area of interest a method of an embodiment of the invention may repeat a clustering process on an expanded or enlarged area, such as for example a double sized area of interest so that for example pixels in the area of interest include the target vessel and at least some other surrounding area can be captured, and a boundary of the target area may be identified.

In some embodiments, if an SDe of even a lowest contour region is out of an acceptable SDe range, an algorithm such as the ΔAD calculation described in block 514, may be used to determine if a target vessel has been for example lost in an image, or if the target vessel takes up an entire area of interest. In an embodiment of the invention, pixel coordinates from a seed area of the image or of a prior image may be added or superimposed as a contour level onto the lowest or other contour region, such as, and referring to FIG. 5A, region 401. The ΔAD algorithm described in block 514 may be executed to compare the contour region with the added contour region of the pixel from the seed area as against the contour region without the added seed area contour region. If the ΔAD algorithm points to the region with the pixel from the seed area, by for exampling returning a lowest derivative for such region, an indication may be deduced that the target vessel has been lost or otherwise does not appear in the contour region and in the area of interest that was clustered. If the ΔAD calculation points to the contour region without the seed point, that may be an indication that the target vessel is in the contour region but that it takes up the entire area of interest.

In block 518 a determination may be made as to whether the segmentation is accurate, such as whether the target vessel has been lost or has failed to appear in, or has been terminated before, a predicted slice. For example, in some cases, a target vessel may not appear in a slice in which it may have been predicted to exist. In some cases such a prediction may be input by a user or may dictated by stored anatomical data for a particular region or vessel. If the segmentation is determined to be inaccurate, by for example a loss of a target vessel in an image, the method may continue to block 520. If the segmentation was deemed satisfactory, such that the target vessel is defined in the image, the method may proceed to block 522.

In block 520, a method of the invention may re-attempt segmentation of the target vessel by returning to a prior slice or image, and re-running the segmentation process described in for example block 508 using a larger area of interest than was used in the previous segmentation attempt. Other methods may be used to find a target vessel that does not appear in a predicted slice.

In some embodiments, the slice at which a second attempted segmentation may be initiated to, for example, find a predicted but not-visible target vessel, may be the slice that immediately preceded the slice wherein the target vessel disappeared. In some embodiments, the prior slice to which the method returns in the repeated segmentation attempt may be two, three or more slices before the slice wherein the target vessel disappeared or wherein the segmentation failed. In some embodiments, if the repeated attempt at segmentation fails to reveal the disappearing target vessel in the later slice, a further segmentation attempt may be initiated with a starting slice that precedes the starting slice in the prior attempt by two or more slices. In some embodiments the earlier slice used to locate a target vessel that disappeared in a current slice, may be a slice or image between the starting slice of segmentation and the slice where the target was lost. In some embodiments, a slice may be selected that is for example three slices prior to the slice where the target was lost. Other increments may be used for re-tracing a lost or disappearing target in prior slices. Earlier and earlier slices may be selected as a starting point for re-attempted segmentations until the lost target is reacquired.

In some embodiments, a disappearance in a current slice of a target vessel, or an SDe outside a pre-defined range may indicate that the vessel has been for example clogged. In block 520, and in some embodiments the method of an embodiment of the invention may re-attempt the segmentation process at a prior or other slice that may be perpendicular or differently angled or on a different plane than the current or prior slice.

In some embodiments, a failure of a segmentation attempt, as may be indicated by for example a disappearance in a current slice of a target vessel or by an SDe outside a pre-defined range, may in some cases be a result of for example a vessel or target structure passing near a high intensity structure such as a bone or larger contrast filled vessel, such that the clustering process did not adequately distinguish between the boundary of a target vessel and the other structure. In such case, an alternative segmentation process may be attempted to extract a region that includes the target vessel from the surrounding or contiguous structures. In an embodiment, such a segmentation process may include construction of a contour gradient map of an image, where such map may be based for example on image intensities of for example several areas in the image. A watershedding process may be executed on the gradients in the constructed map. Following the watershedding process, a seed point may be identified in a section of the image and the clustering process described in blocks 506 and 508, may be repeated on the region that was defined in the watershedding process and that includes the seed point or seed area.

In some cases, a boundary of for example a target vessel may not be apparent even in for example a region that includes the cluster of the low intensity pixels. Furthermore, in some cases, an edge of a target vessel may extend into a part of a lower contour region whose area may not have otherwise been selected for purposes of defining the boundary of the target vessel. In some cases, a vessel or boundary of a vessel may be defined in a segment or part of a contour region that does not include the entire contour region.

In block 522, and further referring to FIG. 5C, a depiction of isolable contour regions in accordance with an embodiment of the invention, a processor may isolate an area bounded by for example two consecutive isolable contour regions, such as for example the area between the edge of region 440 and the edge of region 442. In some embodiments, such area may be designated as the 442-440 region. Pixels in this 442-440 region may be grouped into for example neighborhoods 450, such as for example 8 neighboring pixels or 4 neighboring pixels, based on for example the existence of a shared side 452 between two contiguous pixels such as for example 454 and 456, subject to for example a condition that such two pixels are fully contained with the 442-440 region. In some embodiments, a neighborhood or a set of neighborhoods may include pixels in the 442-440 region that are linked by common or shared sides 452 between contiguous pixels. Each such continuous link or group may constitute a neighborhood 450. In some embodiments neighborhood 450 may include for example pixels that are surrounded for example on all sides by other pixels in the relevant region or that are surrounded by pixels in a region on for example at least two or three sides. Other criteria for inclusion in a neighborhood may be used.

In some embodiments an algorithm that may compares geometric properties of regions, such as for example the ΔA=ΔArea*ΔDistance algorithm described in block 514, may compare a region such as for example the 442-440 region, with a second region that may include that same 442-440 region plus one or more of the neighborhoods such as neighborhood 450A, 450B, and 450C. A result of the function ΔAD{442, 442+A} may be used to determine whether the 450A neighborhood is to be combined with region 442 and considered as defining a boundary of a target vessel. If for example a minimum derivative of ΔAD{442, 402+A} is lower than ΔAD{442} or is lower than for example a pre-defined level, the 450A neighborhood may be included in the boundaries of a target vessel whose boundaries may have otherwise been defined by the edge of region 442. In some embodiments an algorithm that compares geometric properties of regions, such as for example the ΔA=ΔArea*ΔDistance algorithm, may compare neighborhoods such as 460A and 460B in areas between the edges of other contour regions such as 446-444 to determine if such other neighborhoods 460A and 460B are to be included in a boundary of a target vessel that is defined by an edge of such contour region 446. In some embodiments, a determination may be made as to the inclusion of pixels or areas that include pixels in a boundary of a target vessel, on the basis of for example proximity or contiguousness of such pixels to a region or to other pixels, rather than on an image intensity of such pixels.

In block 522 a determination may be made as to whether a target vessel is predicted to have terminated at the slice whose segmentation has been completed. If the target vessel has terminated, or if for example a user has marked the point on the vessel as a point to stop a segmentation, the method may return to for example block 502 where another vessel or seed of a vessel may be selected for segmentation beginning in for example a prior or other slice. If the target vessel has not terminated, the method may continue to block 526.

In block 526, and in some embodiments, a location of a target vessel that is found in an image may be deemed to be or used as the seed area for the segmentation of a next image in the segmentation process, and the segmentation method may be run on the next image or slice. In some embodiments, a segmentation process may end when all of the seeds in all of slices have been subject to a method of segmentation in an embodiment of the invention.

In some embodiments, two or more seed areas may be designated in a single image or slice where for example there is a branch of a target vessel into for example two or more branches. In some embodiments, a method of the invention may segment one or more of such branches, serially or concurrently, and may segment the root and each of the branches. In some embodiments, a plane of the progress of slices may be altered or and the series of images may be segmented in reverse to collect or add missed information that was not segmented in the initial direction or plane.

In some embodiments, a user may indicate that a particular target vessel or branch is not to be segmented beyond a certain distance or beyond a designated point or slice. For example, a user may designate a major vessel as a seed, and may indicate that only certain of the branches of the vessel are to be segmented. An embodiment of a method of the invention may stop the segmentation process of the indicated branches, and continue the segmentation of other branches that are of interest.

In some embodiments, segmentation data may be passed to a post-processing procedure which may for example apply dilation or erosion algorithms, or apply filters such as a Gaussian filter, smoothing filters or filters based on different convolution kernels. Such filters may enhance the display of the segmented data or may remove segmentation artifacts. Other post-processing or display enhancing methods are possible.

Furthermore, in some embodiments, filters may be applied in a pre-processing procedure to decrease noise that may be introduced to the images during the acquisition of these images by the 3D imager. Such smoothing and noise removal can be done by applying a Gaussian filter. Other methods of smoothing and or noise removal may be applied. In some embodiments, filters may be applied in a pre-processing procedure to decrease noise introduced to the images during the acquisition of these images by the 3d imager. Such smoothing and noise removal can be done by applying a Gaussian filter. Other methods of smoothing and or noise removal may be applied.

Embodiments of the invention may be included as instruction such as for example software instructions on for example a computer readable medium such as for example an electronic data storage medium.

It will be appreciated by persons skilled in the art that embodiments of the invention are not limited by what has been particularly shown and described hereinabove. Rather the scope of at least one embodiment of the invention is defined by the claims below.

I claim:

1. A method comprising defining a boundary of a part of a blood vessel in an image of a series of images, said series of images captured by an ex vivo imager, said part of said vessel in said image being free of iodine-based contrast material.

2. The method as in claim 1, comprising designating a seed area in said image.

3. The method as in claim 2, wherein said designating comprises marking an area of said vessel at which to stop a segmentation of said vessel.

4. The method as in claim 2, comprising clustering pixels in a portion of said image containing said seed area, said portion comprising less than all of said image.

5. The method as in claim 1, comprising designating non-uniform ranges of image intensity levels into which pixels are clustered.

6. The method as in claim 1, wherein said defining comprises:
identifying said blood vessel; and
comparing a grayscale scoring of an area in said image to a plurality of stored grayscale scores of samples of said blood vessel.

7. The method as in claim 1, comprising defining a boundary of a plurality of vessels in said image.

8. The method as in claim 1, comprising:
a first clustering of a first plurality of pixels in said image into a first set of clusters; and
a second clustering of a second plurality of pixels into a second set of clusters, said second plurality of pixels being a subset of said first plurality of said pixels.

9. The method as in claim 8, wherein said second clustering comprises selecting a plurality of cluster center means of said second set of clusters, said selection based on an intensity of a pixel in a seed area.

10. The method as in claim 8, wherein said second clustering of said second plurality of pixels into a second set of clusters comprises clustering said second plurality of pixels into more than four clusters.

11. The method as in claim 1, comprising:
a first clustering of a first plurality of pixels in said image into a first set of clusters; and
a second clustering of less than all of said first plurality of pixels into a second set of clusters.

12. The method as in claim 1, comprising:
mapping an isolable contour region of a cluster of pixels, said pixels in said cluster having a range of image intensity ranges, and
selecting from among a plurality of said isolable contour regions having pixels in said range of image intensity levels, a region including a pixel overlapping a pixel in said seed area.

13. The method as in claim 2, comprising:
recording
a coordinate of a pixel within said seed area,
an image intensity of said pixel; and
a designation of said image within said series of images; and
defining a boundary around said area around said pixel, said boundary around said area selected from the group consisting of a boundary box and a convex hole.

14. The method as in claim 1, wherein defining said boundary of said blood vessel comprises defining a boundary of an outer wall of said blood vessel.

15. The method as in claim 1, wherein said defining comprises, selecting a contour level region from among a plurality of contour level regions, by comparing geometric properties of said contour level region to geometric properties of another of said plurality of contour level regions.

16. The method as in claim 15, wherein comparing geometric properties comprises:
calculating a difference between an area of said contour level region and an area of said another of said plurality of contour level regions;
calculating a distance between a mass center of said contour level region and a mass center of said another of said plurality of contour level region;
multiplying said difference between said areas by said distance between said mass centers; and
identifying a derivative of a product of said multiplying.

17. The method as in claim 15, wherein said defining comprises
selecting a group of pixels in a first contour level region;
selecting a second contour level region;
evaluating geometric properties of an area comprising said group of pixels and said second contour level; and
comparing said geometric properties of said area with geometric properties of said second contour region.

18. The method as in claim 17, wherein selecting said group comprises identifying a plurality of pixels in an area between an outer edge of said first contour level region and an outer edge of said second contour level region, where all pixels of said plurality of pixels are contiguous to at least one other pixel of said plurality of pixels.

19. The method as in claim 1, comprising selecting a contour level region from among a plurality of contour level regions based on a comparison of an entropy level of said contour region to an entropy level of other contour level regions.

20. The method as in claim 19, comprising standardizing an entropy level of said contour level region to a log of a number of pixels within said image.

21. The method as in claim 1, comprising selecting a contour level region from among a plurality of contour level regions based on a comparison of an entropy level of said contour region to a pre-defined range of entropy levels.

22. The method as in claim 1, wherein said defining comprises:
defining said boundary of said blood vessel in a first image of said series of images of said vessel; and
detecting that said boundary of said blood vessel does not appear in a second image of said series of images of said blood vessel.

23. The method as in claim 22, comprising:
selecting a third image of said series of images that is between said first image and said second image; and
selecting an area of said third image for a clustering pixels in said area.

24. The method as in claim 23, wherein said selecting said area comprises watershedding an intensity level of said area of said third image, said area overlapping a seed point.

25. The method as in claim 23, wherein said selecting said third image comprises selecting said third image as a predefined number of images from said second image.

26. The method as in claim 23, wherein said selecting said area of said third image comprises selecting said area as being larger than an area in said third image that was selected in a prior segmentation attempt.

27. The method as in claim 23, wherein selecting said third image comprises selecting said third image at an imaging plane that is different than an imaging plane of said second image.

28. The method as in claim 1, wherein said defining comprises grouping into a neighborhood pixels in an area between contour level regions, said pixels comprising less than all of a number of pixels.
